(19) United States
(12) Patent Application Publication
(10) Pub. No.: US 2008/0119745 A1
(43) Pub. Date: May 22, 2008

(54) Manually pressurized electronic sphygmomanometer

(75) Inventors: Paul Yang, Taipei (TW); Shan-Yi Yu, Taipei (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD, CHUNG HO CITY (TW)

(21) Appl. No.: 11/586,549

(22) Filed: Oct. 26, 2006

(51) Int. Cl. A61B 5/02

(52) U.S. Cl. 600/493

(57) ABSTRACT

A manually pressurized electronic sphygmomanometer includes a casing, a display screen disposed on the casing and coupled to an electronic circuit, and a pressure detector in the casing. The pressure detector is coupled to an air current pipeline; the air current pipeline is sheathed onto an end of an interconnect pipe; the interconnect pipe disposed outside the casing has a manual pressurizing device at an end of the interconnect pipe and a pressure release adjusting knob at another end of the interconnect pipe for adjusting the flow of air discharged to the outside. An end of the interconnect pipe is coupled to an airbag, such that when the manual pressurizing device is pressurized, the pressurized air passes through the interconnect pipe to pressurize the airbag, and the pressure detector sends the detected blood pressure to the electronic circuit, and the detected blood pressure values are displayed on the display screen.

### BACKGROUND OF THE INVENTION

1. Field of the Invention

The present invention relates to a manually pressurized electronic sphygmomanometer, and more particularly to a sphygmomanometer that requires medical personnel to manually pressurize the sphygmomanometer and hear the sounds of blood pressures and pulses by a stethoscope to determine and record the systolic/diastolic pressures, and selectively compares such systolic/diastolic pressures with the systolic/diastolic pressures automatically detected by the electronic sphygmomanometer, and uses a display screen instead of mercury to show the systolic/diastolic pressures, so that medial personnel can measure human blood pressures more accurately.

2. Description of the Related Art

Referring to FIGS. 1 and 2 for a general electronic sphygmomanometer, the sphygmomanometer includes a casing 100 for containing an electronic circuit 90, an LCD screen 80 connected to the electronic circuit 90 and disposed on the surface of the casing 100 for displaying and detecting systolic/diastolic pressure values, and a multi-channel interconnect pipe 70 disposed on the casing 100 and connected to a connector 71, and the connector 71 is coupled to an airbag 60 through an air current pipeline 72.

Further, the interconnect pipe 70 is connected separately to a pressure detector 50 and an electric pump 40, wherein the pressure detector 50 is installed on the electronic circuit 90 for detecting a blood pressure, and the interconnect pipe 70 is connected to a pressure releasing device 30 for discharging the pressurized air in the airbag to the outside.

Referring to FIG. 2 for detecting blood pressures, the electric pump 40 pressurizes and sends air to the air current pipeline 72, such that the air passes through the air current pipeline 72 to the airbag 60 worn at a human arm or wrist until the air pressure rises to a predetermined maximum air pressure value, and then the air pressurization stops. When a blood pressure is measured, the air in the airbag 60 flows from the pressure releasing device 30 of the electronic sphygmomanometer to the outside according to a predetermined airflow for a discharge, such that the airbag 60 is released to a predetermined minimum air pressure value, and the pressure detector 50 detects the blood pressures while the air is released slowly, and the detected data of the systolic/diastolic pressures are sent to the electronic circuit 90 for processing, and the detected result is sent to the LCD screen 80 for a display. The pressurization, pressure release and blood detection processes can be achieved and the result is displayed on the screen 80 by simply pressing a start button. However, medical personnel need to know whether or not there is an abnormal pulsation while the heart is supplying blood by using a stethoscope, in addition to simply measuring the systolic/diastolic pressure values when the blood pressures are measured, so as to serve as a reference for determining a patient's condition. Since the pulsation of a certain patient's heart is too weak or the patient's fat is too thick, the heartbeat or pulse rate cannot be detected. Therefore, the electronic sphygmomanometer can simply detect a change of blood pressures and a heartbeat value only, but medical personnel cannot fully trust these readings at all.

If a traditional mercury sphygmomanometer is used for measuring blood pressures, medical personnel need to determine the reading from the mercury column visually, and thus may have misjudgments due to different viewing angles. Furthermore, such mercury sphygmomanometer has a larger volume which is inconvenient to carry and use.

### SUMMARY OF THE INVENTION

In view of the shortcomings of the traditional electronic sphygmomanometers totally relying on an electronic circuit for a pressurization and a pressure release as well as the detection of systolic/diastolic pressures when blood pressures are measured, and such electronic sphygmomanometer cannot let users know whether or not there is an abnormal pulsation while the heart is supplying blood at the time of measuring the blood pressures. Since the pulsation of a certain patient's heart may be too weak, or the patient's fat is too thick, the blood pressures cannot be measured, or the reading of measurements cannot be trusted. Traditional mercury sphygmomanometers also have the drawbacks of a large size and a deviation of visual angle of the mercury column, the inventor of the present invention aimed at the problem of the traditional sphygmomanometers to conduct extensive researches and experiments, and finally invented a manually pressurized electronic sphygmomanometer in accordance with the present invention.

The primary objective of the present invention is to provide a manually pressurized electronic sphygmomanometer that includes a casing, an electronic circuit contained in the casing, a display screen connected to the electronic circuit and disposed on a surface of the casing for displaying and detecting systolic/diastolic pressure values, a pressure detector installed in the casing and connected to the electronic circuit for detecting blood pressure, and an air current pipeline sheathed with an interconnect pipe. The interconnect pipe is disposed outside the casing, and an end of the interconnect pipe is connected to a manual pressurizing device, and another end of the interconnect pipe has a pressure release adjusting knob for manually adjusting the airflow of the discharged air, and another end of the interconnect pipe is connected to an airbag coupled, such that when the manual pressurizing device performs an air pressurization, the air passes through the interconnect pipe to the airbag worn on a human arm or wrist, until the air pressure rises to a predetermined maximum air pressure value and then the air pressurization stops. When a blood pressure is measured, the air in the airbag is released to the outside according to a predetermined airflow until the pressure of the airbag is released to a predetermined minimum air pressure value, such that the pressure detector can detect the pulse of the blood vessels while the air is being released slowly and send the detected blood pressure data to the electronic circuit for processing, and the detected results are sent to the display screen for the display. Medical personnel can determine and record the systolic/diastolic pressure values by hearing the pulse sound of blood vessels from a stethoscope. Such arrangement not only can pressurize the airbag of the sphygmomanometer manually to maintain the using habits of medical personnel, but medical personnel can know if there is an abnormal pulsation while the heart is supplying blood. Further, the results are displayed on the display screen, and thus there will be no visual deviation issue anymore.

Another objective of the present invention is to provide a manually pressurized electronic sphygmomanometer that includes a manual pressurizing device and a support body made of a hard material and covered on a lateral surface of the pressurizing device for facilitating users to hold the pressurizing device and support the casing.

A further objective of the present invention is to provide a manually pressurized electronic sphygmomanometer that includes a press button provided for medical personnel to press in order to record current blood pressures and display the recorded blood pressure values after recording the blood pressures.

Another further objective of the present invention is to provide a manually pressurized electronic sphygmomanometer capable of simultaneously displaying the systolic/diastolic pressure values automatically detected by the sphygmomanometer and the systolic/diastolic pressure values measured and recorded by medical personnel and providing such values for the reference and comparison made by medical personnel.

### BRIEF DESCRIPTION OF THE DRAWINGS

To make it easier for our examiner to understand the objective, shape, assembly, structure, characteristics and performance of the present invention, the following embodiments accompanied with the related drawings are described in details.

FIG. 1 is a schematic view of components of a traditional structure;

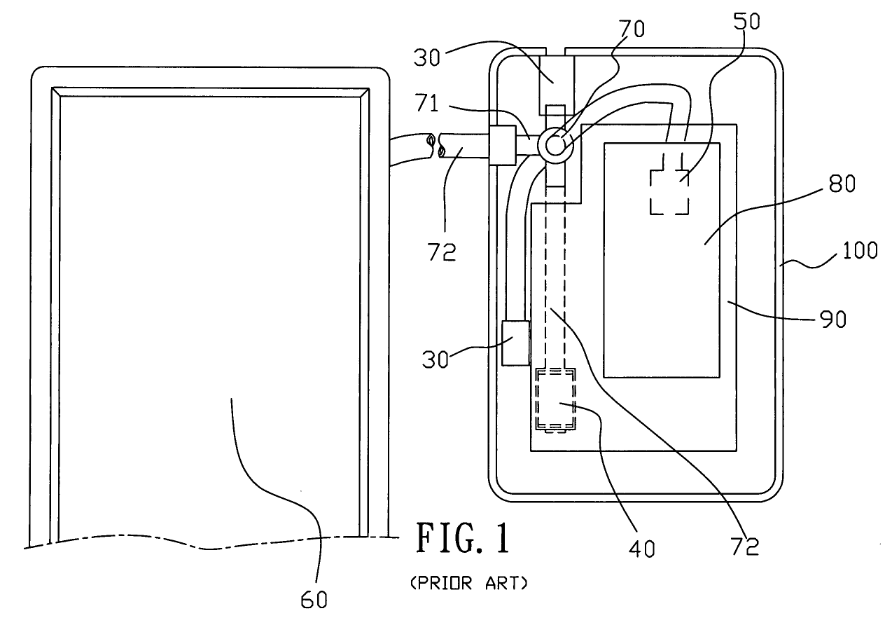

FIG. 2 is a schematic view of an application of a traditional apparatus;

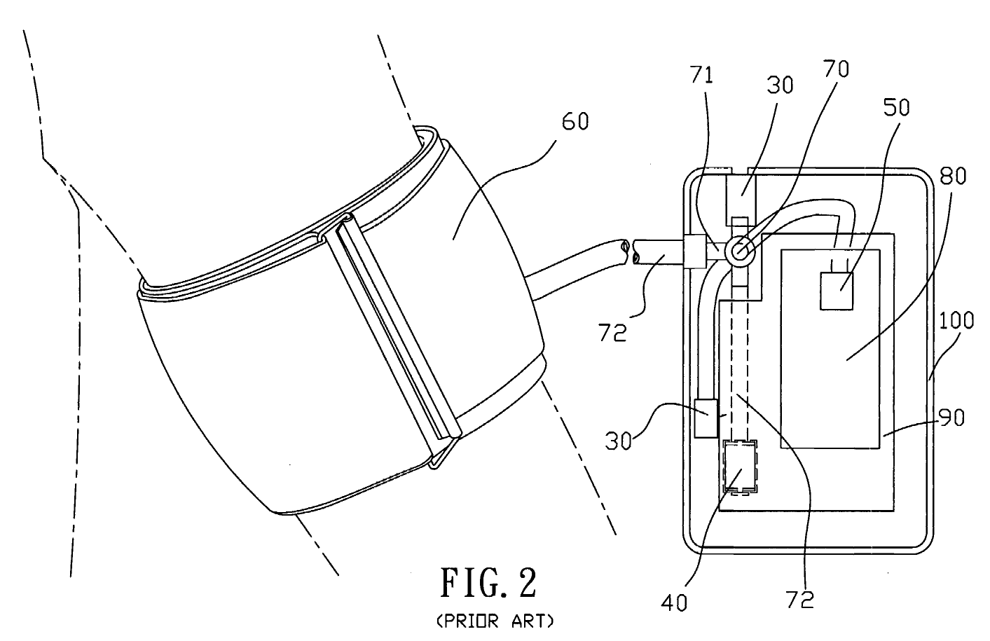

FIG. 3 is a schematic view of components of the present invention; and

FIG. 4 is a schematic view of an application of the present invention.

### DETAILED DESCRIPTION OF THE PREFERRED EMBODIMENTS

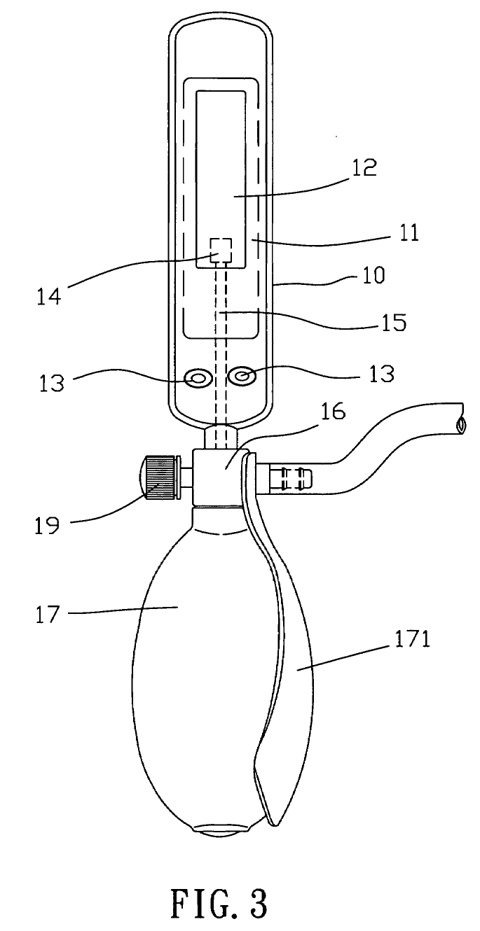

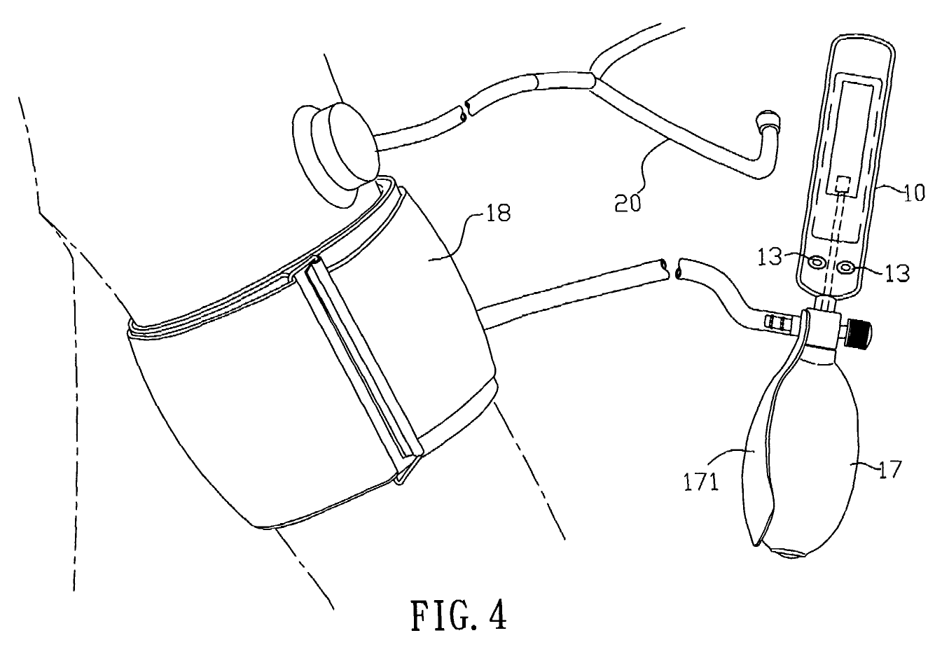

Referring to FIGS. 3 and 4, a manually pressurized electronic sphygmomanometer of the invention comprises a casing 10 for containing an electronic circuit 11, a display screen 12 connected to the electronic circuit 11 and disposed on a surface of the casing and the display screen 12 is an LCD screen in this embodiment for displaying and detecting systolic/diastolic pressure values, and a press button 13 disposed in the casing 10 and connected to the electronic circuit 11 for controlling the ON/OFF of the electronic circuit 11 and recording current blood pressures. The electronic circuit 11 is electrically coupled to a pressure detector 14 for detecting blood pressures, and the pressure detector 14 is coupled to an air current pipeline 15, and the air current pipeline 15 is sheathed with an interconnect pipe 16, and the interconnect pipe 16 is disposed outside the casing 10, and an end of the interconnect pipe 16 has a manual pressurizing device 17, which is an elastic hollow sphere in this embodiment, for adjusting the pressure of the interconnect pipe 16 and the pressure detector 14, and a support body 171 made of a hard material is disposed on a lateral side of the manual pressurizing device 17, and the support body 171 is an iron plate in this embodiment for facilitating users to hold the manual pressurizing device 17 and support the casing 10. Further, other ends of the interconnect pipe 16 are coupled to an airbag 18 and a pressure release adjusting knob 19 for slightly, quickly or manually adjusting the airflow of the air discharged from the airbag 18 to the outside.

When the sphygmomanometer is used, users hold the manual pressurizing device 17 and perform a pressurization with the support of the support body 171, such that air passes through the interconnect pipe 16 to an airbag 18 worn at a human arm or wrist, until the air pressure rises to an appropriate maximum air pressure value and then the pressurization stops. When a blood pressure is measured, the air in the airbag 18 is discharged through the pressure release adjusting knob 19 to the outside, and the pressure release adjusting knob 19 has the function of slightly and quickly adjusting the pressure of the airflow by medical personnel, so that the pressure of the airbag 18 drops to a predetermined minimum air pressure value, and the pressure detector 14 detects the blood pressure while the air is being released slowly, and the detected blood pressure data is sent to the electronic circuit 11 for processing, and the processed results are sent to the display screen 12 for the display. Medical personnel can determine the systolic/diastolic pressure values by using a stethoscope 20 to hear the sounds of blood pressures and pulses. With the operation of the press button 13, the systolic/diastolic pressure values can be recorded. After measurements are taken, medical personnel can selectively press the press button 13 to display the recorded systolic/diastolic pressure values and the systolic/diastolic pressure values automatically detected by sphygmomanometer for reference and comparison or solely display the measured systolic/diastolic pressure values. Such arrangement allows users to perform a pressurization manually and maintain the using habits of the medical personnel, and users can know whether or not there is an abnormal pulsation when the heart is supplying blood, since measurements cannot be taken due to the pulsation of some patient's heart is too weak or the patient's fat is too thick. The invention also can display the results on the display screen 12 without being affected by a deviation of the visual angle.

In summation of the above description, the manually pressurized electronic sphygmomanometer in accordance with the present invention herein provides a simple and easy structure, saves the cost, enhances the performance, overcomes the shortcoming of the prior art, and further complies with the patent application requirements, and thus the invention is duly filed for patent application.

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

1. A manually pressurized electronic sphygmomanometer, comprising:
a casing, having an electronic circuit coupled to a press button, a pressure detector and a display screen, wherein said press button is provided for controlling the ON/OFF of said electronic circuit and an operator determines the systolic/diastolic pressure values by the sounds of blood pressure and pulses, and with the operation of said press button, the systolic/diastolic pressure values are recorded, and said display screen is installed on a surface of said casing surface for displaying blood pressure values, and said pressure detector is provided for detecting a blood pressure and coupled to an air current pipeline;
an interconnect pipe, disposed outside said casing, and sheathed with said air current pipeline in said casing, and an end of said interconnect pipe being coupled to an airbag;
a pressure release adjusting knob, coupled to an end of said interconnect pipe, and capable of adjusting the air flow of a gas in said airbag slightly, quickly or manually; and
a manual pressurizing device, coupled to an end of said interconnect pipe for pressurizing and inflating said airbag.

2. The manually pressurized electronic sphygmomanometer of claim 1, wherein said manual pressurizing device is an elastic hollow sphere.

3. The manually pressurized electronic sphygmomanometer of claim 1, wherein said manual pressurizing device has a support body made of a hard material and covered onto a lateral surface of said manual pressurizing device for facilitating a user to hold said pressurizing device and supporting said casing.

4. The manually pressurized electronic sphygmomanometer of claim 3, wherein said support body is an iron plate.

5. The manually pressurized electronic sphygmomanometer of claim 1, wherein the operator determines the systolic/diastolic pressure values by the sounds of blood pressure and pulses is by using a stethoscope.
